(19) United States
(12) Patent Application Publication
(10) Pub. No.: US 2026/0283964 A1
(43) Pub. Date: Sep. 24, 2026

(54) METHOD FOR MANUFACTURING PHARMACEUTICAL MINI-TABLETS

(71) Applicant: Purdue Research Foundation, West Lafayette, IN (US)

(72) Inventors: Varun Sundarkumar, West Lafayette, IN (US); Zoltan Kalman Nagy, West Lafayette, IN (US); Gintaras V. Reklaitis, West Lafayette, IN (US); Wanning Wang, West Lafayette, IN (US)

(73) Assignee: Purdue Research Foundation, West Lafayette, IN (US)

(21) Appl. No.: 19/475,631

(22) PCT Filed: Feb. 28, 2024
(86) PCT No.: PCT/US2024/017721

§ 371 Date: Oct. 15, 2025

Related U.S. Application Data
(60) Provisional application No. 63/468,685, filed on May 24, 2023.

(51) Int. Cl. A61K 9/20

(52) U.S. Cl. CPC A61K 9/2095; A61K 9/2031; A61K 9/2072

(57) ABSTRACT

A method for manufacturing pharmaceutical mini-tablets with a low dosage and high content of uniformity by employing drop-on-demand three-dimensional printing; a printed mini-tablet manufactured in accordance with the method; and a combination of two or more printed mini-tablets.

### CROSS-REFERENCE TO RELATED APPLICATION

This application claims priority to U.S. provisional patent application No. 63/468,685, which was filed May 24, 2023, and which is hereby incorporated by reference in its entirety.

### STATEMENT OF GOVERNMENT SUPPORT

This invention was made with government support under 2132142 awarded by the National Science Foundation and FD006738 awarded by the U. S. Food and Drug Administration. The government has certain rights in the invention.

### TECHNICAL FIELD

The present disclosure relates to a method for manufacturing pharmaceutical mini-tablets. In particular, the disclosure provides a method for manufacturing pharmaceutical mini-tablets using a drop-on-demand (DoD) three-dimensional (3D) printing system.

### BACKGROUND

This section introduces aspects that may help facilitate a better understanding of the disclosure. Accordingly, these statements are to be read in this light and are not to be construed as admissions about what is or is not prior art.

Many limitations challenge the mass manufacturing of pharmaceutical drugs. Lack of personalization, slow and inefficient supply chains, and drug shortages triggered by product recalls are some drawbacks that have adversely impacted therapeutic outcomes (Lee et al., 2015, Journal of Pharmaceutical Innovation, 10 (3), 191-199). Among these, the inability to produce personalized pharmaceutical products is a particularly pressing problem. Mass-manufactured dosages are often made at a few discrete dose strengths suitable for the broad population. This ‘one-dose-fits-all’ approach leaves patient groups like pediatrics, geriatrics, and those with metabolic or organ dysfunction disadvantaged, as their drug dosing needs differ significantly from the populace (Trenfield et al., 2018, Trends in Pharmacological Sciences, 39 (5), 440-451).

Pediatric medicines make up less than 10% of the overall drug market. Producing drug products for pediatric patients is a challenging problem in pharmaceutical manufacturing. Finding drug products in dosage levels that suit the requirements of children is difficult. Furthermore, drug products made for these patients have many restrictions, which make their production challenging. Children show distinct pharmacokinetic and pharmacodynamic responses to drugs due to variations in metabolic rate, gastrointestinal absorption, and renal function. Children need lower doses of a drug than adults, but as they grow and mature, the dose required through childhood changes a lot. This necessitates having highly flexible drug products available in a broad range of doses, including low doses. Thus, children often require highly flexible low-dose medication. In addition, features like taste masking and ease of swallowability are also vital factors, making them more acceptable to pediatric patients.

Traditionally, pediatric patients have been treated using dosage forms like crushed or split tablets or liquid oral drug products. However, crushing and splitting the tablets risks altering the intended dissolution behavior of the drug product and impacting dosing accuracy as the split subunits may have non-uniform drug amounts. Liquid oral drug products are a popular alternative for medicating children as they can satisfy many of these requirements and are very easy to consume. However, they have several limitations, including many active pharmaceutical ingredients (APIs) are unstable in aqueous media and prone to degradation, and liquids are also susceptible to inaccurate dosing, leading to adverse drug reactions. Compounding pharmacies are also frequently used to make pediatric dosages, especially when commercial tablets are available only at high drug loadings. However, achieving similar release behavior and drug content uniformity across dosages is difficult in compounded products (Zuccari et al., 2022, Pharmaceuticals Vol. 15 (1), 108).

In recent years, mini-tablets have emerged as an attractive dosing solution that can meet these demands. Mini-tablets are small form dosages around 2-4 mm in diameter that can be dispensed individually or in combination. They are manufactured using two known methods: conventional direct compression and hot melt extrusion. Direct compression is challenging due to the poor powder flow properties of many APIs. This limits the preparation of homogeneous formulation blends and the transfer of the blends in uniform quantities into tableting dies. Thus, the production of low-dose medication and small-size dosages is particularly challenging. The hot melt extrusion method is a form of 3D printing in which the API and excipients blend is extruded into filaments and either melted or cut into desired shapes and amounts. This method requires high operating temperatures, which can cause degradation of the API and a lack of control over the polymorphic form of the API.

Thus, there is a need for a manufacturing technique that provides mini-tablets with a low-dose and high degree of content uniformity. It is an object of the present disclosure to provide such a method. This and other objects and advantages, as well as inventive features, will be apparent from the detailed description.

### SUMMARY

Provided is a method for manufacturing a mini-tablet. The method comprises:
i) preparing a drug formulation by mixing an active pharmaceutical ingredient (API) and one or more first excipients;
ii) heating the drug formulation either to obtain a melt-based suspension drug formulation or a melt-based solution drug formulation;
iii) printing a drop of the melt-based drug formulation of step (ii) using a drop-on-demand (DoD) printing system;
iv) collecting a printed drop of the drug formulation in a solidification bath to obtain a solidified drop of the drug formulation; and
v) extracting, washing, and drying the solidified drop of the drug formulation, whereupon the mini-tablet is manufactured.

The one or more first excipients can be selected from polyethylene glycols, polypropyleneglycols, Kolliphor® D-α-tocopherol polyethylene glycol succinate (TPGS), Gelucire® 44/14, food oil, and a combination of two or more thereof. In some embodiments, the polyethylene glycols can be PEG 2000, PEG 600, PEG 4000, or any combination thereof. This excipient can be used in combination with one or more second excipients commonly used in tablets.

Any API can be used. In some embodiments, an amount of an API and one or more first excipients, alone or in combination with one or more second excipients, can be used in a ratio of about 1:1 to about 1:100. In some embodiments, the API can be present in an amount of about 0.01 mg to about 10 mg.

The solidification bath can be prepared using an inert material to solidify mini-tablets. The inert material can be air, liquid nitrogen, silicon oil, or food oil. In some embodiments, the inert material is silicon oil. The printed drop of the drug formulation can solidify at a temperature from about 20° C. to about 60° C. in a solidification bath.

The DoD printing system can dispense the uniform drug formulation to print mini-tablets having content uniformity as stipulated by US Pharmacopeia (USP).

In view of the above, also provided is a printed mini-tablet manufactured by the method. In some embodiments, the mini-tablets obtained have content uniformity with an acceptance value less than 15%, which is the required approval standard by USP. The method can provide low-dose mini-tablets having different release profiles, such as a fast-release or an extended-release. The release profiles of the mini-tablets can be modified by altering one or more first excipients, alone or in further combination with one or more second excipients.

Further provided is a combination of two or more printed mini-tablets manufactured by the method.

### BRIEF DESCRIPTION OF THE DRAWINGS

The present disclosure will be more readily understood from the detailed description of embodiments presented below considered in conjunction with the attached drawings of which:

FIG. 1 shows a schematic of a manufacturing process for pharmaceutical mini-tablets using the drop-on-demand (DoD) three-dimensional (3D) printing system.

FIG. 2A shows the mode of failure of DoD-based mini-tablet manufacturing by the pooling of drops caused by insufficient solidification.

FIG. 2B shows the mode of failure of DoD-based mini-tablet manufacturing by aggregation of drops caused by slow settling speeds.

FIG. 2C shows the mode of failure of DoD-based mini-tablet manufacturing by aggregated formulation drops.

FIG. 3A shows a Pareto set of solutions for the solvent bath design problem.

FIG. 3B shows mini-tablets manufactured by the DoD printing process.

FIG. 4A shows the dissolution profiles of mini-tablets of atorvastatin and lisinopril, manufactured by the DoD printing process, with individual dosages of 1 mg.

FIG. 4B shows the dissolution profiles of mini-tablets of atorvastatin and lisinopril, manufactured by the DoD printing process, with multiple dosages of 10 mg.

FIG. 5 shows the dissolution profiles of mini-tablets of atorvastatin manufactured by the DoD printing process, with extended-release dosages of 10 mg.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the claimed invention is thereby intended.

The term “mini-tablet” refers to small-sized tablets typically having a diameter of 2-4 mm. Mini-tablets are easy to swallow and can be dispensed individually or in combination in flexible doses.

The terms “drug formulation”, “melt-based drug formulation, and “formulation” are used interchangeably.

The term “content uniformity” refers to the variation in the amount of active pharmaceutical ingredient (API), or drug loading, across multiple mini-tablets. High variation in drug loading leads to lower content uniformity. To measure this, the United States Pharmacopeia (USP) recommends using a metric called acceptance value; acceptance values under 15 is one condition to determine an acceptable amount of API variation across dosages.

Drop-on-demand (DoD) is a pharmaceutical three-dimensional (3D) printing technique that builds dosages by printing multiple drops of an API-containing formulation onto a substrate like a capsule or a placebo tablet. It can print a variety of formulations and can produce drug products with a broad range of drug loadings. It also is amenable to emerging developments in pharmaceutical manufacturing like continuous processing and end-to-end operation (Sundarkumar et al., 2022, Journal of Pharmaceutical Sciences, 111 (8), p.p. 2330-2340).

In a view of the above, provided is a method for manufacturing a mini-tablet. The method comprises:
i) preparing a drug formulation by mixing an active pharmaceutical ingredient (API) and one or more first excipients;
ii) heating the drug formulation either to obtain a melt-based suspension drug formulation or a melt-based solution drug formulation;
iii) printing a drop of the melt-based drug formulation of step (ii) using a drop-on-demand (DoD) printing system;
iv) collecting a printed drop of the drug formulation in a solidification bath to obtain a solidified drop of the drug formulation; and
v) extracting, washing, and drying the solidified drop of the drug formulation, whereupon the mini-tablet is manufactured.

In some embodiments, the method for manufacturing mini-tablets comprises printing mini-tablets by using an inkjet-based DoD 3D printing system. The DoD system can provide a continuous manufacturing route for mini-tablets. In this method, the API and excipients are mixed and heated together so that API can be either suspended or dissolved in a molten carrier liquid excipient to obtain a melt-based suspension drug formulation or a melt-based solution drug formulation. The formulation can be printed as drops via a displacement pump nozzle, and each drop can be captured and solidified using a solidification bath to yield a mini-tablet. The method can provide low-dose mini-tablets with a high degree of content uniformity by solidifying individual droplets. The DoD printing can provide drops of drug formulations with precise dosages, which solidify to the mini-tablets that can meet regulatory requirements of USP and United States Food and Drug Administration (USFDA), such as content uniformity and release of dosage.

DoD 3D printing can manufacture mini-tablets for any drug product. Thus, any API can be used. In some embodiments, the API can be a statin, such as atorvastatin, or an ACE inhibitor, such as lisinopril. The amount of the API present in the mini-tablet can range from about 0.01 mg to about 10 mg, such as from about 0.01 mg to 10 mg, 0.01 mg to about 10 mg, or 0.01 mg to 10 mg. Desirably, the amount of the API present in the mini-tablet can range from about 0.1 mg to about 1 mg, such as from about 0.1 mg to 1 mg, 0.1 mg to about 1 mg, or 0. 1 mg to 1 mg.

The one or more first excipients can be selected based on their melting point. The selected excipients can have a melting point below 120° C. In some embodiments, excipients can be selected having a low melting point of about 40° C. to about 70° C., such as about 40° C. to 70° C., 40° C. to about 70° C., or 40° C. to 70° C. Examples of excipients include, but are not limited to, polyethylene glycols, such as PEG 2000, PEG 600, and PEG 4000, polypropyleneglycols, Kolliphor® D-α-tocopherol polyethylene glycol succinate (TPGS), Gelucire®, food oil, such as coconut oil, lard, or a combination of two or more thereof. This excipient can be used in combination with one or more other second excipients that are commonly used for tablet manufacturing. Changing the excipients can affect the acceptance value (AV) for content uniformity (see Table 2).

The amounts of API and one or more first excipients, alone or in combination with one or more second excipients, used are in a ratio of about 1:1 to about 1:100, such as about 1:1 to 1:100, 1:1 to about 1:100, or 1:1 to 1:100.

The drug formulation can be prepared by mixing an API and one or more first excipients in the required amounts, which can be decided based on the dosage of the mini-tablet. The drug formulation can be heated at a temperature from about 40° C. to about 70° C., such as from about 40° C. to 70° C., 40° C. to about 70° C., or 40° C. to 70° C. to obtain either a melt-based suspension formulation or a melt-based solution formulation. Thus, the method avoids the challenges of handling powder forms of APIs or their drug formulations.

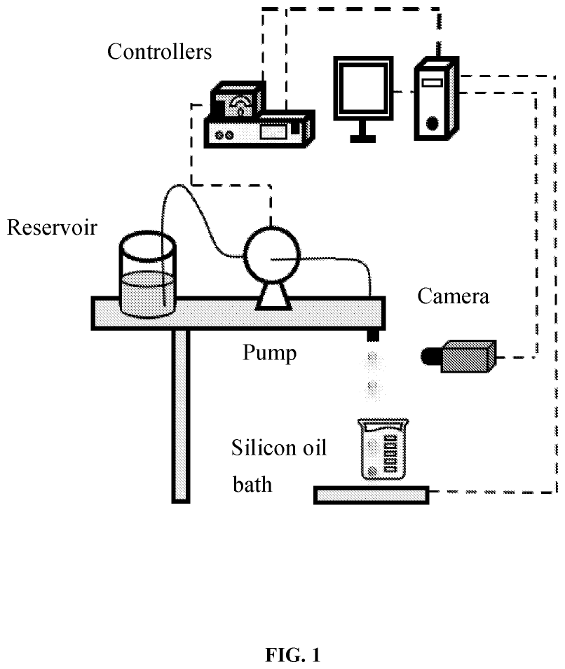

FIG. 1 shows a schematic of the DOD 3D printing system. The printing system comprises i) a reservoir that can hold the formulation ink, i.e., the drug formulation, for printing with constant agitation to ensure concentration homogeneity; and ii) a high-precision positive displacement pump that dispenses an accurate volume of the formulation ink through a nozzle in the form of a drop (FIG. 1). It can print much larger drops (with volumes in the microliter range) compared to typical inkjet systems (drop volumes in the nano or picoliter range), which allows it to produce drug products faster.

The method comprises capturing and solidifying printed drops of the drug formulation. The printed drops can be captured and solidified using a solidification bath. The bath can be an inert material bath, allowing the printed drops to solidify without deformation. The printed drops can be solidified at a temperature from about 20° C. to about 60° C., such as from about 20° C. to 60° C., 20° C. to about 60° C., or 20° C. to 60° C. The drops can be extracted, washed with a lighter silicon fluid such as hexamethyldisiloxane (HMDSO), and dried to yield mini-tablets. HMDSO can be used as a washing fluid for the mini-tablets as it is a lighter inert silicon oil that can remove heavier oil adhering to the tablets.

Thus, the DoD system of the disclosure can avoid the filament cutting and extrusion steps required by a known hot melt extrusion method and can generate droplets with uniform volumes, which in turn provides dosages with high content uniformity.

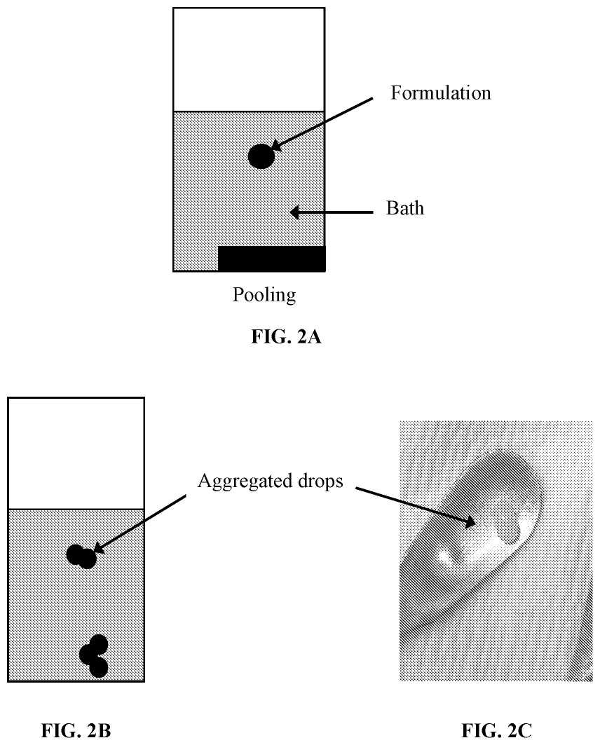

The preparation of the solidification bath plays a critical role in avoiding the failure of manufacturing mini-tablets. The manufacturing of mini-tablet can fail if there is insufficient solidification, which causes the pooling of drops (see FIG. 2A), and slow settling speeds, which cause aggregation of drops (see FIG. 2B).

The method for manufacturing mini-tablets further comprises preparing a solidification bath that can capture and solidify dosages uniformly. The preparation of the solidification bath can consist of i) selecting a material for the solidification bath and ii) determining the properties of the solidification bath.

The material for the bath can be selected based on its compatibility with formulation, solidification efficiency, and effect on drop uniformity. In some embodiments, the material can be inert, such as air, liquid, nitrogen, silicon oil, or food oils, such as vegetable oil, sunflower oil, or peanut oil. The inert material does not interact with either the API or excipients. Other important factors that can be considered for selecting material can be compatibility with drug formulation, solidification efficiency, and effect on drop uniformity. Desirably, the inert material is silicon oil. Silicon is a safe inactive ingredient approved by the USFDA. It can avoid the breakage of droplets either during or after solidification, and it can provide droplets with a broad range of properties such as viscosity, surface tension, and density.

The properties that improve solidification efficiency can be solidification time and bath chamber height. In order to achieve high throughput, quick solidification of drops is desirable. For a compact production unit, it is preferable to minimize the amount of solvent required in the bath, which is proportional to the height of the settling chamber, assuming that the cross-sectional area of the chamber is constant. Factors affecting these metrics are settling velocity and heat duty, i.e., the amount of heat to be extracted from the drop during solidification. A multi-objective optimization can be formulated and solved to achieve an optimal trade-off preparation of the solidification bath as described in the examples herein below.

The mini-tablets can be manufactured in different shapes. In some embodiments, the tablets are in spherical shape. The mini-tablets can meet the pharmacopeial requirements. In some embodiments, the diameter of mini-tablets can range from about 2.0 mm to about 4.0 mm (such as from 2.0 mm to 4.0 mm). The dosage of mini-tablet can be from about 0.01 mg to about 10 mg, such as from about 0.01 mg to 10 mg, 0.01 mg to about 10 mg, or 0.01 mg to 10 mg. Desirably, the dosage of mini-tablet can be from about 0.1 mg to about 1 mg, such as from about 0.1 mg to 1 mg, 0.1 mg to about 1 mg, or 0. 1 mg to 1 mg.

The mini-tablet can be uncoated or coated, dispersible, fast-release, or extended-release. The fast-release (e.g., formulations 3 and 4 of Table 1) or extended-release (e.g., formulations 8 and 9 of Table 1) profile of the mini-tablet can be achieved by modifying one or more first excipients, alone or in further combination with one or more second excipients. FIG. 5 demonstrates extended-release formulations 8 and 9 of atorvastatin, with 10 mg of the drug being released in about 8 hours (e.g., 8 hours) and about 12 hours (e.g., 12 hours), respectively. These formulations can differ from fast-releasing formulations 3 and 4.

Further, provided is a combination of two or more printed mini-tablets manufactured by the method described herein above.

Drug products have many critical quality attributes, such as content uniformity, residual solvent content, or dissolution behavior. For 3D-printed drug products, content uniformity is a very important metric since precise dosing is one of the key features of 3D printing. The content uniformity, along with uniformity in shape and weight of the mini-tablets, and dissolution properties of mini-tablets were analyzed. The results were assessed based on the acceptance criteria specified by USP and the FDA.

TABLE 1
Formulations

| | Drug loading | | |
|---|---|---|---|
| | per mini- | Formulation | Formulation |
| Variations | tablet | (API + excipient) | number |
| Drug loading | 1 mg | atorvastatin + PEG 2000 | 1 |
| | 0.1 mg | atorvastatin + PEG 2000 | 2 |
| Excipients | 1 mg | atorvastatin + PEG blend | 3 |
| | | (2000: 25%, 600: 75%) | |
| | 1 mg | atorvastatin + PEG blend | 4 |
| | | (4000: 10%, 600: 90%) | |
| | 1 mg | atorvastatin + Kolliphor ® TPGS | 5 |
| | 1 mg | atorvastatin + Gelucire ® 44/14 | 6 |
| API (and | 1 mg | atorvastatin + Gelucire ® 44/14 | 6 |
| formulation | 1 mg | lisinopril + Gelucire ® 44/14 | 7 |
| type) | | | |
| Dissolution | 1 mg | atorvastatin + PEG blend | 3 |
| behavior | | (2000: 25%, 600: 75%) | |
| | 1 mg | atorvastatin + PEG blend | 8 |
| | | (2000: 70%, 600: 30%) | |
| | 1 mg | atorvastatin + PEG blend | 9 |
| | | (2000: 90%, 600: 10%) | |

Table 2 illustrates the content uniformity tests of mini-tablet formulations nos. 1-9 of Table 1. The tested formulations were seen to possess acceptable content uniformity scores with acceptance value (AV)<15%, as stipulated by USP. The varying physical properties of excipients, such as viscosity, surface tension, and density, can alter the drop formation properties of the formulation.

TABLE 2
Content uniformity measurements for mini-tablets.

| | | | | | | | Acceptance |
|---|---|---|---|---|---|---|---|
| | Shape | | Weight | | Drug loading | | value |
| | Mean | RSD | Mean | RSD | Mean | RSD | (10 units) |
| Formulation | (mm) | (%) | (mg) | (%) | (mg) | (%) | (%) |
| 1 | 2.84 | 1.22 | 16.27 | 2.18 | 1.018 | 2.44 | 3.65 |
| atorvastatin + | | | | | | | |
| PEG 2000 (1 mg) | | | | | | | |
| 2 | 3.14 | 1.04 | 21.27 | 1.54 | 0.107 | 2.31 | 12.84 |
| atorvastatin + | | | | | | | |
| PEG 2000 (0.1 mg) | | | | | | | |
| 5 | 3.13 | 4.30 | 15.25 | 4.65 | 1.125 | 4.73 | 14.32 |
| atorvastatin + | | | | | | | |
| Kolliphor ® | | | | | | | |
| TPGS | | | | | | | |
| 6 | 3.03 | 2.13 | 16.21 | 4.35 | 1.031 | 3.76 | 10.00 |
| atorvastatin + | | | | | | | |
| Gelucire ® | | | | | | | |
| 44/14 | | | | | | | |
| 7 | 3.01 | 1.91 | 16.33 | 3.11 | 0.925 | 5.40 | 15.02 |
| lisinopril + | | | | | | | |
| Gelucire ® | | | | | | | |
| 44/14 | | | | | | | |

These results showed that the mini-tablets manufactured using the DoD printing system can meet current regulatory standards for content uniformity and were at par with other mini-tablet production techniques in this regard. The method has several advantages: 1) processing API as a suspension or liquid formulation avoids many of the powder handling challenges; 2) the DoD system generates droplets with uniform volumes, which, in-turn, translates into dosages with high content uniformity as errors associated with filament cutting and extrusion are eliminated; 3) the designed solidification bath provides fault-free tablet generation with no pooling or aggregation of drops; and 4) the DoD platform facilitates the incorporation of emerging innovations in pharmaceutical manufacturing like continuous processing, real-time quality assurance, automation, and process analytical technology.

It will be appreciated by persons skilled in the art that the present disclosure is not limited by what has been particularly shown and described herein above. Rather the scope of the present disclosure includes both combinations and sub-combinations of the various features described hereinabove as well as variations and modifications which would occur to persons skilled in the art upon reading the specification and which are not in the prior art.

### Examples

### Materials

a) Instruments Ultra-pressure liquid chromatography (UPLC, Waters) system with an ultraviolet (UV) detector was used to determine drug loading in mini-tablets (260 nm for atorvastatin and lisinopril).
Nikon Eclipse E600 microscope was used to image the mini-tablets.
Vankel VK 7000 dissolution system with an auto-sampling manifold was used to conduct the dissolution test.
Cary 60 UV spectrophotometer was used to determine the amount of drug dissolved at different time points.
Ohaus Explorer analytical balance was used to weigh the mini-tablets.
Electric heaters designed by the Amy Instrumentation Facility at Purdue University were used.

### b) Chemicals

All chemicals were purchased from commercial suppliers.
Xiameter PMX-200 silicone fluid 1000 cSt (dimethicone) (Dow chemical company) Hexamethyldisiloxane (Thermo Scientific),
Polyethylene glycol (PEG) with average molecular weights of 600, 2000, and 4000 (Tokyo Chemical Industry, TCI, America),
Gelucire® 44/14 (Gattefossé), and Kolliphor® TPGS (BASF Pharma), Lisinopril (TCI America) and atorvastatin (Dr. Reddy's Laboratories),
Potassium phosphate monobasic and sodium hydrogen phosphate (Thermo Scientific) are used to make the dissolution medium,
Deionized water (3.48 g/L).

C) Abbreviations

| | |
|---|---|
| API | Active pharmaceutical ingredient |
| DoD | Drop-on-demand |
| USP | United States Pharmacopeia |
| FDA | United States Food and Drug Administration |
| PEG | Polyethyleneglycol |
| HMDSO | Hexamethyldisiloxane |
| Silicon oil | Xiameter PMX 200 (dimethicone) |
| UPLC | Ultra pressure liquid chromatography |
| AV | Acceptance value |
| RSD | Relative standard deviation |
| MW | Molecular weight |

Before designing the process, the excipient and other materials were selected based on their characteristics. i) PEG is an excipient widely used in drug products and acts as a dissolution enhancer. It melts at ~65° C. and is a solid at room temperature. ii) silicon oil is used as a bath solvent to solidify the printed droplets as it is inert and does not interact with either the API or PEG. This class of compounds is also very flexible and is available with a large range of physical properties. iii) HMDSO is used as a washing fluid for the mini-tablets as it is a lighter inert silicon oil that can remove heavier oil adhering to the tablets.

### A] Preparation of the Inert Solvent Bath

The solvent bath was prepared to capture and solidify drops of drug formulation. The bath design consisted of two parts: selecting the materials for the bath and determining the desired properties of the bath. For material selection, the most important considerations were compatibility with formulation, solidification efficiency, and effect on drop uniformity. Developing an inert bath was advantageous in ensuring compatibility with various APIs and excipients. Commonly available inert materials, air, liquid nitrogen, and silicon oil (polydimethylsiloxane or dimethicone) were considered as suitable candidates. Among these, air was readily available and could be easily introduced into the system. However, its solidification efficiency was low and required an air column of several meters for complete solidification. Moreover, the relatively high settling velocity of drops caused them to break upon impact. On the other hand, liquid nitrogen has excellent solidification efficiency, but the solidified drops condensed moisture from the air, which affected compatibility with formulation components. Furthermore, it had been observed that formulation droplets tend to fracture when printed into liquid nitrogen, creating non-uniform dosage units. Silicon oil was selected based on three features: it is approved as a safe inactive ingredient by the FDA, it avoids breakage of droplets either during or after solidification, and it is available in a broad range of properties such as viscosity, surface tension, and density. This, in turn, allowed for tuning the drop solidification process as needed. Thus, silicon oil was chosen as the bath material for the manufacturing process.

For designing optimal bath properties, two metrics governing solidification efficiency was considered: solidification time and bath chamber height. In order to achieve high throughput, quick solidification of drops was desirable, and to build a compact production unit it was preferable to minimize the amount of solvent required in the bath (this is proportional to height of the settling chamber, assuming that the cross-sectional area of the chamber is constant). Factors affecting these metrics were settling velocity and heat duty (amount of heat to be extracted from the drop during solidification). These objectives were, however, not completely aligned, low solidification time ‘tₛ’ was achieved by having high settling velocity ‘vₜ’ for the droplets (higher convective heat transfer), but this would consequently increase the chamber height ‘I_{c}’. Thus, a multi-objective optimization was formulated and solved to identify an optimal trade-off design. Two constraints were also imposed to avoid the most common failure modes of the bath: insufficient cooling leading to formulation pooling in the vessel and very slow settling leading to drop aggregation (FIGS. 2A and 2B). The design variables chosen for this problem were the bath solvent's viscosity ‘η’ and ‘T_{f}’ the final temperature to which the drop is cooled. The dual objective design problem was posed as follows:

$${( min )}_{Tf , η} ( {l}_{c} , {t}_{s} )$$

$$st : 23 ° C . \leq {T}_{f} \leq 35 ° C . , 0.1 Pas \leq η \leq min ( 10 , {t}_{drops} / 18 {gd}_{drops} ( {p}_{MT} - {p}_{S i} ) ) Pas$$

The first constraint ensured that the drop was fully solidified before it reached the vessel bottom, preventing pooling. 23° C. was the lowest temperature the mini-tablet could attain (with a room temperature bath), and 35° C. was the temperature where the mini-tablet solidified fully (the melting range for excipients used is 40-60° C.). The second constraint ensured that successive drops had a sufficient gap between them to avoid aggregation. The time and distance between successive drops, t_{drops}, and d_{drops}, respectively, were specified by the designer based on the characteristics of the DoD printer being used. For this system, values for t_{drops} and d_{drops} were set to 5 seconds and d_{MT} (one drop diameter), respectively. These parameters can vary across manufacturing systems due to factors such as satellite drop formation and flow currents in the settling chamber. The constraint is comprised of two parts η<t_{drops}/18 gd_{drops} (ρ_{MT}-ρ_{Si}) and 0.1<η<10 Pas. The first part was obtained from the settling velocity of drops required in the chamber to avoid aggregation, vₜ>d_{drops}/t_{drops}, substituting the expression for settling velocity yields η<5/18 gd_{MT}(ρ_{MT}-ρ_{Si}). Bounds of 0.1 Pas and 10 Pas were added to n to limit the solution space, baths with viscosities lower or higher than these bounds required either large settling chambers that won't physically fit in the printing system (order of meters) or required very long solidification times (order of multiple minutes) respectively. I_{c} and tₛ are calculated as follows:
1. System properties: d_{MT}=4 mm, ρ_{MT}=1124 kg/m³, C_{pMT}=2135 J/kgK, k_{MT}=0.31 W/mK, Tᵢ=70° C., ρₛᵢ=950 kg/m³, k_{Si}=0.15 W/mK, C_{PSi}=1250 J/(mol K), T_{bath}=23° C., ΔHₘₑₗₜ=100 kJ/kg, T_{melting}=45° C., t_{drops}=5 sec, d_{drops}=d_{MT}.
2. Terminal settling velocity of the droplets was calculated. vₜ=(gd²_{MT})/18η(ρ_{MT}−ρ_{f}).
3.Computed heat transfer rate (assume convection dominated). h=Nu k/d, Nu=2+(0.4Re^{0.5}+0.06Re^{2/3})Pr^{0.4}, Re=(ρ_{f} vₜ d)/η.
4. Calculated solidification time tₛ=(ρ_{MT} VC_{P})/hA ln((Tᵢ−T_{b})/(T_{f}−T_{b}))+ΔHρV/hA(Tₘ−T_{b}) and l_{c}=vₜ tₛ.
5. Verified if convection-driven heat transfer assumption is valid, i.e., if hd/k>>1.

This problem was solved by constructing a Pareto front using the Non-dominated Sorting Genetic Algorithm II (NSGA2) in the Python library ‘pymoo’ (Blank and Deb, 2020, IEEE Access, 8,89497-89509). Derivation for Nu and log mean temperature difference expressions were used. (Incropera et al., 2007. Fundamental of Heat and Mass Transfer Sixth Edition). A few other assumptions were also made in this calculation: for computing the heat duty and solidification time (e.g. step 4), the drops were assumed to be composed of pure excipient. This approximation was acceptable as the API loading was low (<7%). Other factors like internal fluid circulation in the solidifying drop and the increase in temperature of the silicon oil bath were also neglected. The diameter of the settling chamber needed to be designed such that there were no interactions between the wall and the settling droplet. Empirically, wall effects on particle settling velocity can be ignored if the ratio of vessel diameter to particle diameter is greater than 10, dᵥₑₛₛₑₗ/d_{MT}>10 (Winterberg and Tsotsas, 2000, AIChE Journal, 46 (5), 1084-1088). A settling chamber with a diameter of 30 mm, which is approximately 10 times the size of the mini-tablets produced, was chosen.

### B] Manufacturing of Mini-Tablet

The APIs selected were atorvastatin and lisinopril, which are among the drugs in the priority list defined in the Best Pharmaceuticals for Children Act (BPCA, 2017-18). The choice of excipients, polyethylene glycol mixture (PEG), Gelucire® 44/14, and Kolliphor® TPGS, was based on their low melting point (in the range of 40-70° C.), assuring that the mixture was in solid state at room temperature. Various API-excipient pairs were formulated (see Table 1) to observe the manufacturing process of dosages with variations in drug loading, excipient composition, and release profiles. The formulations were prepared by adding API and excipient in required amounts to a glass vial, with API concentration equal to that desired in the final mini-tablet. The vial contents were then heated while stirring on a hotplate for 30 minutes to obtain a homogeneous formulation. The positive displacement pump, the tubes, and the nozzle were heated using electric heaters to prevent solidification inside the DoD apparatus. The printed drops were collected in batches in the silicon oil bath and, once solidified, were manually extracted. The printed drops were washed with a lighter silicon fluid, such as a hexamethyldisiloxane (HMDSO), to remove any adhered silicon oil, and dried in a fume hood for 20 minutes. The resulting mini-tablets were clean and ready-to-use.

### C] Content Uniformity and Dissolution Behavior of Mini-Tablets

Measurements of content uniformity and dissolution behavior were made for the manufactured mini-tablets. Each formulation was tested using 25 dosage units to determine content uniformity and 5 dosage units to determine dissolution behavior. An additional 10 dosage units were used for comparison of dissolution behavior with a commercial drug product. The consistency in shape was determined by imaging each dosage unit with a microscope and measuring its diameter using the ImageJ software package. Consistency in weight was evaluated by calculating weight variation across mini-tablets. Drug loading was measured by dissolving the mini-tablets in a methanol or methanol-water mixture (for lisinopril formulation) and analyzing them using a UPLC system. The dissolution behavior of mini-tablets was measured using a Vankel VK 7000 dissolution system with an auto-sampling manifold. The mini-tablets were dissolved in 900 mL of a buffer with a pH of 6.8, and samples were collected at regular intervals and analyzed on a UV spectrophotometer (Atorvastatin: 242 nm, Lisinopril: 212 nm) to quantify the amount of API dissolved.

### Results

### A] Preparation of the Inert Solvent Bath

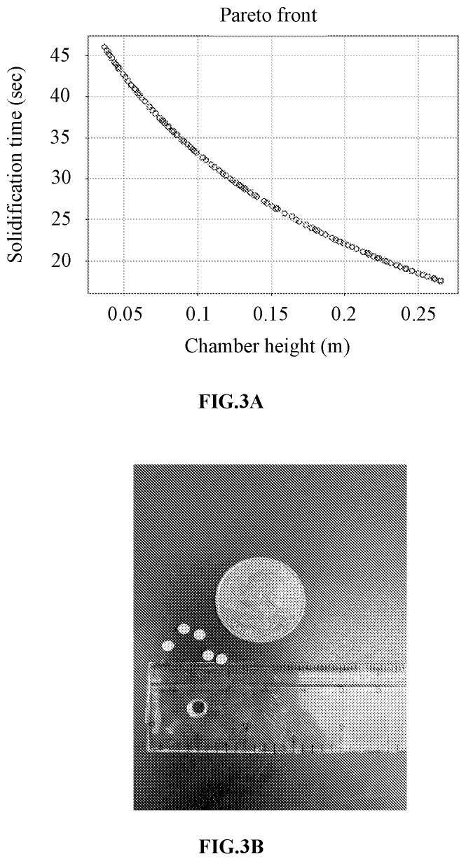

The multi-objective optimization was carried out using NSGA2 algorithm. The objective was to obtain a set of Pareto-optimal solutions for chamber height and solidification time, from which a desirable solution can be selected. FIG. 3A shows that a smooth Pareto front was obtained in the region of interest explored. The curve also followed an expected trend: lower solidification time can be achieved by increasing the rate of convective heat transfer, which in turn can be accomplished by increasing the settling velocity. However, this led to an increase in the chamber height, as the drop must be supported in the bath for a longer distance. Among these non-dominated points, the condition having a bath viscosity of 0.95 Pas was chosen for operation since it is closest to a silicon oil available at hand. The optimal bath design was obtained as follows: η=0.95 Pas, Tₜ=34.9° C., l_{c}=6.5 cm, and tₛ=40.8 sec. The final dosage temperature was close to the constraint value, as this minimizes heat duty and solidification time. This framework provided a general approach for designing the solidification bath, a key component of the DoD-based mini-tablet manufacturing process. Additional constraints or objectives can be incorporated into this method to determine the optimal bath for various scenarios.

### B] Content Uniformity of Mini-Tablets

Mini-tablets were manufactured for all formulations listed in Table 1 using the prepared silicon oil bath, with fault-free tablet generation and no pooling or aggregation of drops (FIGS. 2A and 2B). Table 2 illustrates the results of the content uniformity tests. Most tested formulations were seen to possess acceptable content uniformity scores (Acceptance Value, AV<15%) as stipulated by USP. Additionally, the variation in shape and weight of mini-tablets was also seen to be less than 5% relative standard deviation (RSD).

The acceptance value (AV) scores vary amongst formulations, with the best performance observed for the formulation generating 1 mg mini-tablets with atorvastatin dissolved in PEG. Changing the drug loading to 0.1 mg of atorvastatin changes the AV score. However, this was not due to increased variability in drug loading, as RSD scores in both cases were similar. Instead, the AV changed due to the mean drug loading being further away from the target (7% higher). This showed that some powder handling challenges can still affect DoD operation, particularly in the accurate preparation of the formulation. The observing formulations 1, 5, and 6 in Table 2, showed that changing the excipients also affects the AV scores. This was because varying physical properties such as viscosity, surface tension, and density can alter drop formation properties in these formulations. This may lead to a higher incidence of undesirable phenomena like satellite drop formation, which increases variation in drug loading across dosages. Thus, tuning both the physical properties of the formulation and printing conditions in the DoD system was crucial to achieving accurate drop formation (Sundarkumar et al., 2022b, AIChE Journal 69 (4), e17990).

Formulations 6 and 7 in Table 2 showed that changing the type of formulation (lisinopril formed a suspension in Gelucire® 44/14) also affected the content uniformity of dosages manufactured. Suspensions were known to enable the delivery of higher drug loadings, as they are not limited by the solubility limit of API in the excipient carrier. Thus, in order to achieve acceptable content uniformity with suspension formulations, the range of operating conditions must be constrained to avoid the formation of satellite drops. This can be accomplished by adjusting either the formulation composition affecting droplet viscosity, surface tension, and density or the printer parameters affecting droplet size, ejection speed, and production rate. Overall, these results showed that the mini-tablets manufactured by the DoD printing system can meet current regulatory standards for content uniformity and are at par with other mini-tablet production techniques in this regard.

### C] Dissolution Behavior of Mini-Tablets

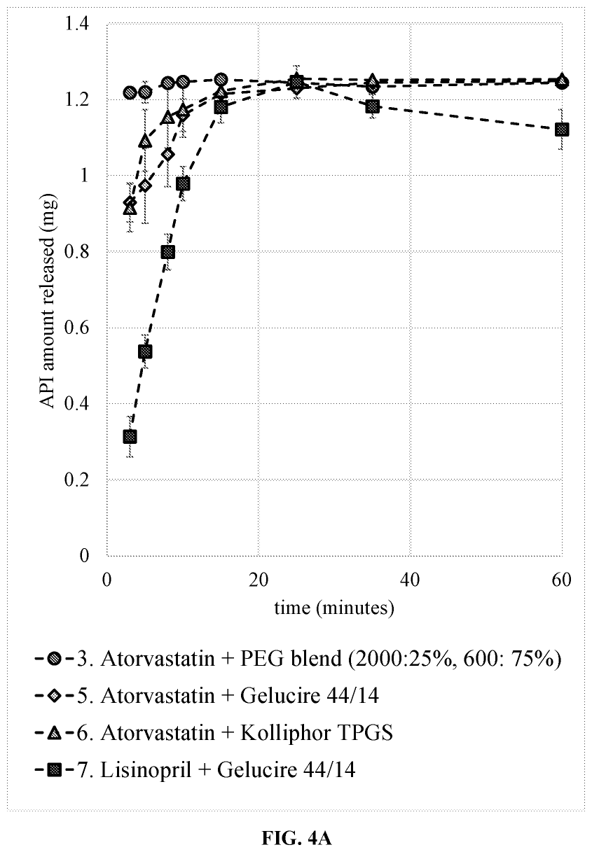

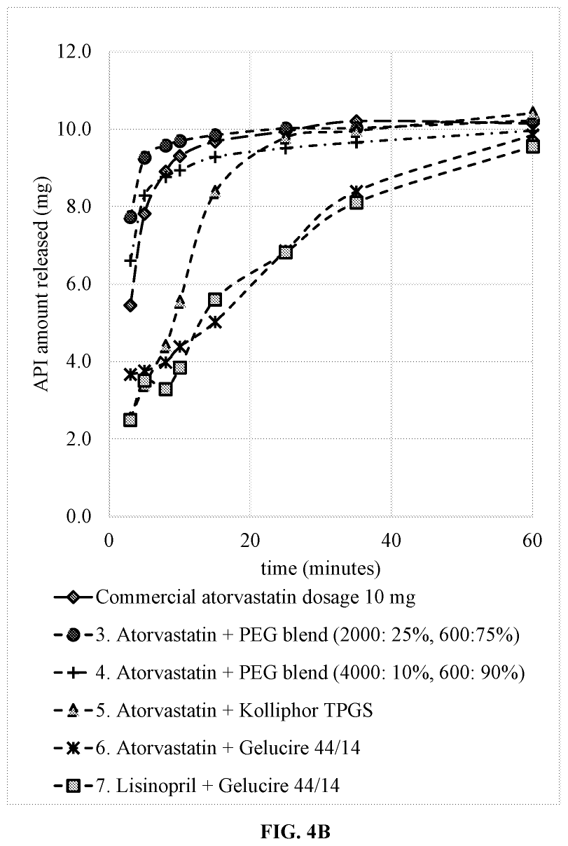

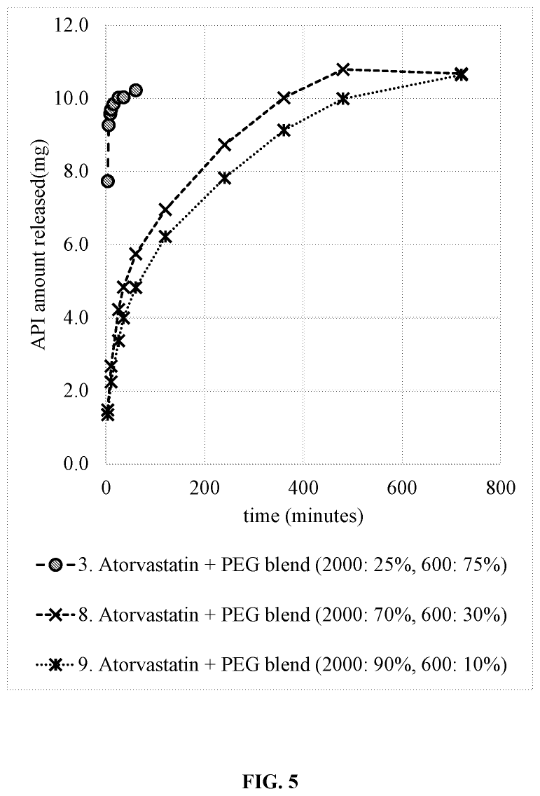

The dissolution behavior of manufactured dosages was investigated for three mini-tablet dispensing scenarios: as single units, as multiple units (and comparing with commercial drug products), or as extended-release units. For single unit mini-tablets (FIG. 4A), it was observed that all formulations dissolved rapidly, with complete drug release occurring within 20 minutes. Formulation 3 (Atorvastatin+PEG blend (2000: 25%, 600: 75%)) showed the fastest release, with the entire dosage dissolving in under 3 minutes. The variance in the amount of drug dissolved was low, and it decreased with time. All mini-tablets were seen to deliver a dose of approximately 1.2 mg. This slightly higher value could be attributed to a shift of the calibration curve at very low concentrations of API, as concentrations less than 1 mg/L of API were measured in this experiment. Content uniformity measurements by UPLC and dissolution tests with multiple dosages confirmed that the mini-tablet contains approximately 1 mg of the drug.

In the case of multiple mini-tablets (FIG. 4B), the release profiles of 10 dosage units dispensed together (resulting in a 10 mg dose) were evaluated and compared with a commercial atorvastatin tablet of the same dose strength (10 mg). To compare dissolution profiles, FDA recommends using f1 and f2 similarity scores, which should range between 0-15 and 50-100, respectively, indicating a good match. Formulations 3 and 4, with similarity scores of 7.4, 92.5, and 5.16, 97.2, respectively, closely resemble the release shown by the commercial dosage. It was seen that changing the excipient leads to a change in the release profile. This was because excipients have different rates of dissolution in water, thus affecting the rate at which API was exposed to the dissolution medium. Furthermore, dissolution profiles of multiple mini-tablets were observed to differ from those of their single counterparts. This is due to differences in the surface area of dosages exposed to the dissolution media. When multiple units were dispensed, they tended to cluster together at the bottom of the dissolution vessel, leading to a net reduction in the surface area exposed. Formulations 6 (solution-melt) and 7 (suspension-melt) showed that changing the type of formulation has little effect on the dissolution behavior. This goes against the intuitive expectation as suspension-based mini-tablets contain larger API particles that dissolve slowly. This effect could have been counteracted by the higher hydrophilicity and dissolution rate of lisinopril compared to atorvastatin, leading to approximately equal dissolution rates for both formulations. Evaluating the manufacturing process for different excipients was important to demonstrate flexibility in case a change in excipient is necessitated due to patient allergies, taste preferences, or API incompatibility.

Formulations 8 and 9 demonstrated extended release of atorvastatin (FIG. 5), with 10 mg of the drug being released in 8 and 12 hours, respectively. These formulations differ from fast-releasing formulations 3 and 4) solely in the composition of the PEG blend used. The strategy behind using a PEG blend in mini-tablets was to exploit the distinct physical properties of different molecular weight (MW) grades of PEG. PEGs with lower MWs (like PEG 600) were softer and dissolved quickly due to their smaller molecular size, whereas PEGs with larger MWs, such as PEG 2000 and 4000, were harder and dissolved slowly. In mini-tablets, the higher MW PEGs served as binding agents and lower MW PEG acted as disintegrating agents, which released API quickly. Thus, using a PEG formulation rich in low MW components yielded fast dissolution, while a PEG formulation rich in high MW components resulted in an extended release. This property thus provided a convenient mechanism that can allow for tuning the release behavior of mini-tablets as desired.

Overall, these experiments demonstrated that mini-tablets manufactured using the DoD printer have low variability in their release, can match the dissolution behavior of commercial products, can be produced with various excipients and formulation types, and can be tailored to possess quick or extended dissolution properties.

As used herein, the following terms and phrases shall have the meanings set forth below. Unless defined otherwise, all technical and scientific terms used herein have the same meaning as commonly understood to one of ordinary skill in the art.

The term “about” can allow for a degree of variability in a value or range, for example, within 10%, within 5%, or within 1% of a stated value or of a stated limit of a range.

The term “substantially” can allow for a degree of variability in a value or range, for example, within 90%, within 95%, or within 99% of a stated value or of a stated limit of a range.

The terms “a,” “an,” or “the” are used to include one or more than one unless the context clearly dictates otherwise. The term “or” is used to refer to a nonexclusive “or” unless otherwise indicated. In addition, the phraseology or terminology employed herein, and not otherwise defined, is for the purpose of description only and not of limitation. Any use of section headings is intended to aid the reading of the document and is not to be interpreted as limiting. Further, information that is relevant to a section heading may occur within or outside of that particular section. The terms “including” and “having” are defined as comprising (i.e., open language).

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

It is intended that the scope of the present methods and apparatuses be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

1. A method for manufacturing mini-tablets which method comprises:
i) preparing a drug formulation by mixing an active pharmaceutical ingredient (API) and one or more first excipients;
ii) heating the drug formulation either to obtain a melt-based suspension drug formulation or a melt-based solution drug formulation;
iii) printing a drop of the melt-based drug formulation of step (ii) using a drop-on-demand (DoD) printing system;
iv) collecting a printed drop of the drug formulation in a solidification bath to obtain a solidified drop of the drug formulation; and
v) extracting, washing, and drying the solidified drop of the drug formulation, whereupon the mini-tablet is manufactured.

2. The method of claim 1, wherein one or more first excipients are a polyethylene glycol, a polypropyleneglycol, Kolliphor® D-α-tocopherol polyethylene glycol succinate (TPGS), Gelucire®, a food oil, or a combination of two or more thereof.

3. The method of claim 1, wherein the polyethylene glycol is PEG 2000, PEG 600, PEG 4000, or any combination thereof.

4. The method of claim 1, wherein the one or more first excipients can be used in combination with one or more second excipients.

5. The method of claim 1, wherein one or more first excipients, alone or in combination with one or more second excipients, and API are used in a ratio of about 1:1 to about 1:100.

6. The method of claim 5, wherein the amount of the API used is about 0.01 mg to about 10 mg.

7. The method of claim 1, wherein the solidification bath is prepared using an inert material.

8. The method of claim 7, wherein the inert material is air, liquid nitrogen, silicon oil, or food oil.

9. The method of claim 8, wherein the inert material is silicon oil.

10. The method of claim 1, wherein the printed drop of the drug formulation is solidified at a temperature from about 20° C. to about 60° C.

11. The method of claim 1, wherein the DoD printing system dispenses the drug formulation uniformally to print mini-tablets having content uniformity as stipulated by US Pharmacopeia.

12. The method of claim 11, wherein the mini-tablets have content uniformity with an acceptance value of less than 15%.

13. The method of claim 1, wherein modification of one or more first excipients, alone or in further combination with one or more second excipients, results in the manufacture of a mini-tablet with a fast-release or an extended-release profile.

14. A printed mini-tablet manufactured by the method of claim 1.

15. A combination of two or more printed mini-tablets manufactured by the method of claim 1.
